(19) United States
(12) Patent Application Publication
(10) Pub. No.: US 2026/0000891 A1
(43) Pub. Date: Jan. 1, 2026

(54) METHOD AND SYSTEM FOR ELECTRICAL STIMULATION BASED ON PHYSIOLOGICAL PARAMETER FEEDBACK REGULATION

(71) Applicant: Fasikl Incorporated, DALLAS, TX (US)

(72) Inventor: Zhi Yang, DALLAS, TX (US)

(21) Appl. No.: 19/329,625

(22) Filed: Sep. 16, 2025

(30) Foreign Application Priority Data
Aug. 18, 2025 (CN) 202511150351.5

(51) Int. Cl. A61N 1/36; A61N 1/02; A61N 1/04

(52) U.S. Cl. CPC A61N 1/36031; A61N 1/025; A61N 1/0456; A61N 1/0472

(57) ABSTRACT

The present disclosure provides a method and a system for electrical stimulation based on physiological parameter feedback regulation. The method includes: continuously acquiring action data and corresponding tremor manifestation data in daily activities of a user; calculating an average tremor level of the user based on the tremor manifestations; identifying a plurality of actions exhibiting tremor manifestations deviating from the average tremor level, and constructing an action set; constructing a continuous action sequence based on the action set; and guiding the user to execute the continuous action sequence while respectively applying electrical nerve stimulation with a plurality of candidate stimulation parameter sets, determining a first electrical stimulation parameter set that satisfies the conditions that the tremor level of the Category I actions decreases, and the tremor level of the Category II actions does not rise, and performing subsequent electrical stimulation using the first electrical stimulation parameter set.

### TECHNICAL FIELD

The present disclosure belongs to the field of electrical stimulation therapy devices, and particularly relates to a method and a system for electrical stimulation based on physiological parameter feedback regulation.

### BACKGROUND

In treatment of neuromotor disorder-related diseases, electrical nerve stimulation, as a non-pharmaceutical intervention means, has been widely applied to alleviate symptoms of diseases such as Parkinson's disease and essential tremor. For such technologies as the electrical nerve stimulation, electrical stimulation signals are typically applied to specific neural regions via transcutaneous electrodes or implanted electrodes to regulate aberrant neural activity patterns, thereby alleviating clinical manifestations including muscle tremor and bradykinesia.

For most systems of electrical nerve stimulation in the prior art, fixed parameters are employed for stimulation output, such as a stimulation frequency, a pulse width, and a current intensity, which typically will not be adjusted dynamically after initial assessment or clinical setting. Some relatively advanced systems, although having parameter adjustment capabilities, mainly rely on manual observation of clinical manifestations or tremor responses of patients to implement the parameter adjustment process, and healthcare personnel perform limited parameter tuning based on empirical determination. These systems lack real-time performance and precision, thus failing to adapt to persistent fluctuations in individual states.

Notably, tremor manifestations are closely related to specific actions of patients, and the tremor amplitudes, tremor frequencies, and probabilities of incidence may vary significantly when individuals execute different actions. Certain actions may induce intense tremor, whereas other actions may suppress or mask tremor occurrence. Consequently, setting stimulation parameters only based on tremor data acquired in static postures or singular postures, without consideration of a role of action states in tremor manifestations, will severely compromise the efficacy of an electrical stimulation strategy in a practical dynamic activity scenario, which may even lead to adverse effects or failure risks. Therefore, the role of action factors in tremor responses should be fully considered for optimization and feedback regulation of electrical stimulation parameters to enhance therapeutic precision and stability.

### SUMMARY

To solve the problems in the prior art, the present disclosure provides a method for electrical stimulation based on physiological parameter feedback regulation, and the method includes the following steps:
providing a wearable electrical nerve stimulation system, where the system is configured to deliver electrical stimulation, and acquire action parameters and tremor level parameters;
continuously acquiring action data and corresponding tremor manifestation data in daily activities of a user;
calculating an average tremor level of the user based on the tremor manifestations;
identifying a plurality of actions exhibiting tremor manifestations deviating from the average tremor level, and constructing an action set, where the action set includes Category I actions having a tremor level significantly higher than the average tremor level, and Category II actions having a tremor level significantly lower than the average tremor level;
constructing a continuous action sequence based on the action set, where the continuous action sequence includes at least one Category I action and at least one Category II action; and
guiding the user to execute the continuous action sequence while respectively applying electrical nerve stimulation with a plurality of candidate stimulation parameter sets, determining a first electrical stimulation parameter set that satisfies the conditions that the tremor level of the Category I actions decreases relative to the daily level, and the tremor level of the Category II actions does not rise relative to the daily level, and performing subsequent electrical stimulation according to the first electrical stimulation parameter set.

Further, user action signals are acquired via an inertial measurement unit (IMU), temporal features of the IMU action signals are classified, and actions are classified based on the temporal features.

Further, tremor level data are determined via a sliding window; a width of a statistical window is dynamically regulated based on a tremor signal fluctuation; the width of the statistical window is reduced when it is detected that the tremor signal fluctuation exceeds a first preset value; and the width of the statistical window is increased when the tremor signal fluctuation is below a second preset value.

Further, the tremor level of each action category is smoothed by applying a moving average method or median filtering, and an action is incorporated into the action set only when the action exhibits a consistent deviating trend in a plurality of independent cycles.

Further, the continuous action sequence includes alternately arranged Category I actions and Category II actions.

Further, the Category I actions and the Category II actions are screened based on an action proficiency and a muscle strength distribution of the user.

Further, an application order of candidate parameters is dynamically regulated based on a Bayesian optimization strategy.

Further, the plurality of candidate stimulation parameter sets are determined by a cloud-based AI model in combination with historical data from a plurality of users.

The present disclosure further provides a system for electrical stimulation based on physiological parameter feedback regulation, and the system includes the following modules:
a wearable electrical nerve stimulation system, where the system includes an electrical stimulation module, an action acquisition module, a tremor level acquisition module, and a processing module;
the action acquisition module is configured to continuously acquire action data in daily activities of a user;
the tremor level acquisition module is configured to synchronously acquire tremor manifestation data corresponding to the action data;
the processing module is configured to calculate an average tremor level of the user based on the tremor manifestations, identify a plurality of actions exhibiting tremor manifestations deviating from the average tremor level, and construct an action set that includes Category I actions having a tremor level significantly higher than the average tremor level, and Category II actions having a tremor level significantly lower than the average tremor level;
the processing module is further configured to construct, based on the action set, a continuous action sequence including at least one Category I action and at least one Category II action;
the electrical stimulation module is configured to respectively apply electrical nerve stimulation with a plurality of candidate electrical stimulation parameter sets when the user executes the continuous action sequence, and determine a first electrical stimulation parameter set; the first electrical stimulation parameter set satisfies the conditions that the tremor level of the Category I actions decreases, and the tremor level of the Category II actions does not rise; and
the electrical stimulation module is further configured to perform electrical nerve stimulation according to the first electrical stimulation parameter set during subsequent treatment.

Further, user action signals are acquired via an inertial measurement unit (IMU), temporal features of the IMU action signals are classified, and actions are classified based on the temporal features.

Further, tremor level data are determined via a sliding window; a width of a statistical window is dynamically regulated based on a tremor signal fluctuation; the width of the statistical window is reduced when it is detected that the tremor signal fluctuation exceeds a first preset value; and the width of the statistical window is increased when the tremor signal fluctuation is below a second preset value.

Further, the tremor level of each action category is smoothed by applying a moving average method or median filtering, and an action is incorporated into the action set only when the action exhibits a consistent deviating trend in a plurality of independent cycles.

Further, the continuous action sequence includes alternately arranged Category I actions and Category II actions.

Further, the Category I actions and the Category II actions are screened based on an action proficiency and a muscle strength distribution of the user.

Further, an application order of candidate parameters is dynamically regulated based on a Bayesian optimization strategy.

Further, the plurality of candidate stimulation parameter sets are determined by a cloud-based AI model in combination with historical data from a plurality of users.

According to the present disclosure, a dynamic association between tremor and specific actions is established by incorporating an action-dependent tremor feature identification mechanism, which effectively enhances the pertinence and reliability of electrical stimulation parameter selection. Through identifying fluctuation features of tremor in different action states and constructing the continuous action sequence as a feedback assessment template, stimulation effects may be assessed accurately in a plurality of scenarios, such that stimulation failure or adverse effects caused by local optima are avoided.

In the present disclosure, comprehensive assessment and optimal screening of stimulation parameters in a plurality of action states are further achieved by applying the plurality of candidate stimulation parameter sets in a process of executing the continuous action sequence and recording tremor variations in real time, such that the individualized adaptability and generalization capability of stimulation control are enhanced. This strategy overcomes a response latency inherent in conventional fixed parameter or manual adjustment methods, and allows the electrical stimulation to become more precise, more dynamic, and safer.

### BRIEF DESCRIPTION OF THE DRAWINGS

In order to explain the technical solutions in embodiments of the present disclosure or the prior art more clearly, the drawings to be used in the embodiments will be briefly introduced below. Obviously, the drawings described below are only some embodiments of the present disclosure. For those of ordinary skill in the art, other drawings may be obtained based on these drawings without making creative efforts.

FIG. 1 is a flowchart of a method of the present disclosure.

FIG. 2 shows a collaborative optimization and feedback mechanism of a cloud-based AI model.

FIG. 3 shows a module structure of a system of the present disclosure.

### DETAILED DESCRIPTIONS OF THE EMBODIMENTS

The present disclosure is described below in a preferred manner in combination with the drawings and the specific embodiments.

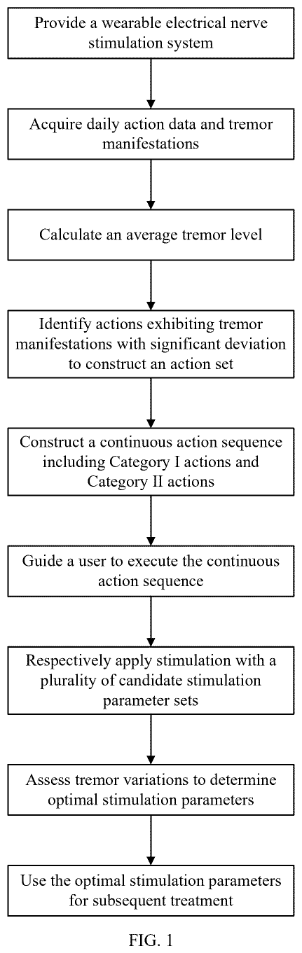

As shown in FIG. 1, in an embodiment, according to a method for electrical stimulation based on physiological parameter feedback regulation of the present disclosure, an electrical stimulation therapy for personalized closed-loop intervention control is achieved by acquiring physiological parameter data of an individual user in daily activities and stimulation processes, analyzing correlation between tremor manifestations and action features, and dynamically regulating electrical nerve stimulation parameters based on feedback patterns, and the electrical stimulation therapy is suitable for alleviating symptoms of and optimizing therapeutic effects for neuromotor disorder-related diseases such as essential tremor and Parkinson's disease.

The present disclosure is based on a wearable electrical nerve stimulation system, where the system is configured to deliver electrical stimulation, acquire action parameters and tremor level parameters, and achieve the above effects through the following steps:
continuously acquiring action data and corresponding tremor manifestation data in daily activities of a user;
calculating an average tremor level of the user based on the tremor manifestations;
identifying a plurality of actions exhibiting tremor manifestations deviating from the average tremor level, and constructing an action set, where the action set includes Category I actions having a tremor level significantly higher than the average tremor level, and Category II actions having a tremor level significantly lower than the average tremor level;
constructing a continuous action sequence based on the action set, where the continuous action sequence includes at least one Category I action and at least one Category II action; and
guiding the user to execute the continuous action sequence while respectively applying electrical nerve stimulation with a plurality of candidate stimulation parameter sets, determining a first electrical stimulation parameter set that satisfies the conditions that the tremor level of the Category I actions decreases relative to the daily level, and the tremor level of the Category II actions does not rise relative to the daily level, and performing subsequent electrical stimulation according to the first electrical stimulation parameter set.

In the above solution, an individualized action-tremor relationship model is established by continuously acquiring action data and corresponding tremor manifestations in daily activities of the user. The system is configured to automatically identify patterns of significant fluctuations in the tremor level during specific actions, and classify such actions as tremor-sensitive actions. Subsequently, in the process of executing such continuous tremor-sensitive actions by the user, different electrical stimulation parameter combinations are applied, and an actual intervention effect of each parameter set is determined through dynamic variations in tremor feedback. The finally selected electrical stimulation parameters are those effective for tremor control in a plurality of action states without inducing additional tremor responses, and thus optimization of an individualized electrical stimulation solution based on action scenario differences is achieved.

Compared to conventional electrical stimulation solutions based on feedback regulation in single states, this solution takes into full account the coupling features between actions and tremor, and allows the selected parameters to better align with an actual physiological state of the patient by screening stimulation parameters from the dynamic action sequence. By verifying suppression effects in tremor-amplifying actions and confirming no adverse interference in tremor-reducing actions, the selected parameters exhibit higher universality and stability, and the precision, safety, and long-term adaptability of the stimulation therapy are effectively improved.

To further explain the above solution, the principles, embodiments, and technical effects of each step in the above solution are described below in detail.

Tremor, as a manifestation of neuromotor disorders closely associated with specific actions, exhibits significant variations in intensity and frequency under different actions. Electrical stimulation methods in prior art mostly rely on fixed actions or static states as a basis for assessment, thereby failing to comprehensively reflect tremor responses in real-life scenarios. Consequently, acquiring tremor manifestations based on action variations and dynamically regulating electrical stimulation parameters according to the feedback information sourced from the acquired tremor manifestations constitute a critical pathway for enhancing an individualized level and a therapeutic effect precision. The present disclosure, based on this technical principle, integrates action information with tremor responses to establish a closed-loop feedback mechanism by providing a wearable system, and thus achieves precise, dynamic, and adaptive electrical nerve stimulation parameter regulation.

In the present disclosure, the electrical nerve stimulation system refers to a wearable device capable of outputting electrical stimulation signals, and includes an electrical stimulation module, a signal acquisition module, and a data processing unit.

The signal acquisition module includes an inertial measurement unit (IMU) configured to acquire action data and tremor manifestations of a user, and the IMU typically includes an accelerometer and a gyroscope, and is capable of acquiring triaxial motion information.

The tremor manifestations refer to involuntary oscillatory behaviors exhibited by the user during action execution, typically characterized by indices such as an amplitude and a frequency of high-frequency components in acceleration signals.

The action data refer to spatial motion state information of limbs or a trunk of a user, including direction, velocity, acceleration, and posture.

The electrical stimulation module is configured to apply preset electrical signals to target neural regions via transcutaneous electrodes, and the IMU is configured to acquire action data of the user in natural living states.

The signal acquisition module further includes an electromyographic signal (EMGs) acquisition module configured to assist in determining muscle activation states, and the data processing module is configured to perform feature extraction and analysis on the above data.

During operation of the system, the IMU continuously acquires motion trajectories of the user in different action states, and extracts high-frequency components from tremor signals through a signal processing means such as high-pass filtering. The system records each action and the corresponding tremor manifestations, and calculates an average level of the tremor manifestations after a quantity of the recorded data reaches a set threshold.

In a preferred embodiment, a wavelet packet decomposition method is employed to extract tremor signals, and decompose under multi-scale conditions triaxial acceleration or angular velocity signals acquired by the IMU to extract high-frequency tremor components. The wavelet packet decomposition method, by constructing a multi-layered wavelet packet decomposition architecture, is configured to decompose raw IMU signals into a plurality of frequency-band signals and extract tremor features from each sub-band, and thus achieves precise isolation and analysis of tremor components of different frequency ranges. Typically, essential tremor mainly concentrates in a range of 4-12 Hz, whereas a Parkinsonian tremor frequency primarily resides in a range of 3-7 Hz. Consequently, by selecting an appropriate wavelet basis function (e.g., Daubechies wavelet) and decomposition levels, frequency-domain representational capability for different tremor types may be effectively enhanced, and interference from low-frequency postural variations and high-frequency noise is suppressed simultaneously. Compared to conventional time-domain filtering, this strategy exhibits enhanced frequency-domain-focused capability, and is capable of more accurately extracting tremor components in environments with complex action states and mixed tremor, improving tremor level assessment precision, and providing more stable input signals for subsequent electrical stimulation feedback control.

In a preferred embodiment, the system includes an action labeling module configured to perform pattern classification based on temporal features of IMU signals to achieve automatic identification and standardized annotation of user actions. The action labeling module, by training supervised learning models such as convolutional neural networks (CNN), long short-term memory networks (LSTM), or Transformer architectures, is configured to perform feature extraction and classification on acceleration and angular velocity data acquired in a continuous motion process of the user, further automatically identify typical action states such as arm raising, first clenching, object holding, and static posture, and match corresponding action category labels with the acquired tremor data. During implementation, the system may acquire multi-session action training data from the user, build an action dictionary library, and continuously optimize an identification accuracy of a classification model. In practical applications, the system automatically completes action segmentation and annotation when the user executes the continuous action sequence. This approach eliminates annotation lag and unstable precision arising from traditional methods relying on manual observation or manual labeling, ensures precise mapping between actions and tremor manifestations, and facilitates action correlation analysis for stimulation effects and personalized parameter screening.

In a preferred embodiment, the electrical stimulation parameters include an electrical stimulation frequency, a pulse width, and a stimulation intensity. The system, by employing a sliding window mechanism, is configured to assess the average tremor suppression effect and tremor fluctuation of each parameter combination under continuous action conditions. The sliding window mechanism is configured to perform statistical analysis on tremor responses of each parameter combination within the time window by setting fixed or variable-length time intervals, and extract indices such as an average tremor amplitude, a variation rate, and an instantaneous peak value, to quantify an impact of electrical stimulation parameters on the tremor level in different action phases. Throughout execution of a complete continuous action test cycle, the system records tremor responses corresponding to each action label under each parameter set, and comprehensively assesses a tremor suppression capability in high-tremor actions and a stability in low-tremor actions, thereby determining optimal stimulation parameters. This strategy significantly enhances the adaptability of the stimulation solution in a dynamic motion process, ensures that selected stimulation parameters not only exhibit superior performance in single actions but also maintain stable intervention effects in diverse action combinations, and improves the robustness and versatility of therapeutic strategies.

To achieve targeted and dynamic regulation of electrical stimulation parameters, it is necessary to establish an individualized tremor baseline of the user in natural states, which serves as a reference standard for assessing stimulation effects. Due to the action correlation and individual difference of tremor, the tremor levels of different users exhibit fluctuations in different time periods and actions. Absence of a unified baseline will induce bias in stimulation parameter selection. Through long-term statistical analysis of tremor manifestations acquired in daily activities of the user, the overall tremor levels in a plurality of actions and states are extracted, and an average tremor level is calculated, which provides an objective basis for subsequently identifying actions exhibiting significant tremor variations and assessing stimulation response intensity, and thus supports personalized feedback regulation processes.

The system is configured to continuously acquire tremor manifestation data in different action scenarios during routine daily activities of the user through the IMU. To ensure representativeness and stability of the acquired data, a statistical cycle is set as a plurality of days or a plurality of complete activity cycles. Within the statistical cycle, the system is configured to perform preprocessing on acquired tremor amplitude signals, including band-pass filtering to suppress low-frequency postural interference and high-frequency noise.

The processed tremor signals are divided into a plurality of time windows. Within each time window, tremor features including maximum amplitude value, root mean square (RMS) value, and dominant spectral peak frequency are extracted as tremor manifestations for the time segment.

The system is configured to perform an averaging operation on tremor amplitude values extracted from all time windows, and calculate the average tremor level.

To further enhance the robustness of this index, the system may combine weighted averaging of different feature indices or median filtering, to avoid signal distortion caused by aberrant tremor peaks.

In a preferred embodiment, the system incorporates a tremor stability factor configured to dynamically determine whether tremor data in different time intervals are statistically stable, which enhances an accuracy of average tremor level estimation. The tremor stability factor is obtained by calculating a standard deviation of tremor amplitude values within a sliding window, a tremor amplitude dataset extracted from the same time window serves as input, and a fluctuation degree is measured using the standard deviation. When the standard deviation is below a preset stability threshold of the system, it is determined that the tremor signals within the time period exhibit minor fluctuations, and the data demonstrate sufficient representativeness and reproducibility and may be incorporated for calculating the average tremor level of the user. Conversely, if the standard deviation exceeds the preset threshold, it indicates that tremor manifestations fluctuate severely within the time period due to presence of unstable factors such as action transition, environmental interference, or postural abnormality. Such data are flagged and excluded from the calculation of the average tremor level. This approach may effectively suppress offset in overall tremor estimation induced by sporadic anomalies or local perturbations, such that calculated results exhibit high robustness and clinical reference value. During implementation, a stability threshold is automatically set based on pre-training data of target groups, or manually adjusted by physicians according to diagnostic experience. Following incorporation of the tremor stability factor, the tremor level outputted from the system is more consistent and stable, which is helpful for obtaining reliable feedback in the electrical stimulation parameter regulation process.

In a preferred embodiment, the system is configured to group the tremor manifestation data of the user according to action categories, and respectively calculate average tremor levels under different action categories to construct a multi-baseline tremor model, thereby enhancing sensitivity to and distinguishing capacity for action factors in tremor analysis. Specifically, based on an action identification module, the system annotates each segment of IMU signal data as corresponding action labels, respectively calculates the average tremor amplitude, tremor frequency, and tremor fluctuation indices with respect to tremor data under each label, and generates a statistical tremor baseline set divided by action categories. Such classification approach is configured to distinguish inherent tremor manifestations in different action states such as “cup holding”, “arm raising”, “static posture”, and “walking”, and is further utilized for constructing a multi-baseline tremor assessment model. Incorporation of the multi-baseline model enables the system not to rely on a singular tremor mean value as a reference standard during subsequent tremor monitoring and stimulation effect analysis; instead, the system may perform differential analysis and deviation degree determination based on the baseline value corresponding to the current action, such that determination errors of the tremor level induced by action transition are effectively avoided. During implementation, the system supports continuous updating of the baseline model, performs adaptive training in combination with long-term user data acquired, and dynamically optimizes mapping relationships between action categories and tremor distribution, which further enhances a stimulation parameter regulation precision under multi-scenario and multi-posture conditions.

In a preferred embodiment, the system may employ an adaptive window length algorithm to dynamically regulate a width of a statistical window based on fluctuation conditions of tremor signals. When severe fluctuations or high short-term variation rates are detected in the tremor signals, the width of the statistical window is reduced to enhance temporal resolution and responsiveness to high-frequency transients. Conversely, when the tremor signals are relatively stable or exhibit a slow variation trend, the width of the statistical window is increased appropriately to improve signal-to-noise ratio and anti-interference capability. This approach ensures both timeliness and stability, and is helpful to enhance the accuracy and robustness of tremor level estimation.

In a specific example, the system acquires tremor manifestation data when a patient wearing a device of the present disclosure executes standardized actions during five consecutive days, including “horizontal arm raising”, “wrist rotating, and “fist clenching and releasing”. During a data processing phase, the system extracts the tremor amplitude RMS values within successive time windows of 10-second duration, and totally 7,200 sets of tremor amplitude data are obtained. After removal of outliers and filtering are performed on all data, the system calculates the average tremor level of the user as 0.43 g (where g represents an acceleration unit). During subsequent stimulation testing, the tremor levels of all actions under different stimulation parameters are compared with this average value, which serves as a reference basis for determining tremor suppression effects. The finally selected stimulation parameters exhibit a tremor reduction trend across a plurality of consecutive tests, and the system completes personalized parameter generation.

To achieve action-specific optimization of electrical nerve stimulation parameters, it is necessary to identify specific actions inducing significant variations in the tremor level during execution. Since the tremor level may exhibit an increased, decreased, or stabilized trend in different actions, extraction and classification of such differential actions are helpful for constructing a representative action set for subsequent stimulation parameter testing and screening. Specifically, by identifying actions exhibiting tremor manifestations with significant deviation from the average tremor level, an action set including Category I actions and Category II actions is constructed, and is configured to simulate multi-scenario conditions of stimulation responses, such that the distinguishing capability of the feedback mechanism is enhanced.

The system is configured to analyze tremor manifestation data corresponding to different actions based on the previously calculated average tremor level of the user. The system may set a distinguishing threshold configured to identify actions that induce tremor manifestations deviating significantly from the average tremor level. Specifically, the system calculates the average value of tremor amplitude indices in the process in which the user repeatedly executes a given action.

The given action is determined as a Category I action when the average tremor amplitude of the given action is higher than a preset proportional threshold of the average tremor level of the user (e.g., higher by 15%). The given action is determined as a Category II action if the average tremor amplitude of the given action is lower than a preset proportional threshold of the average tremor level (e.g., below 85%). All satisfied actions are respectively classified into a Category I action set and a Category II action set.

Finally, the system constructs an action set including at least one Category I action and at least one Category II action, which are configured to simulate neural responses in different tremor states during subsequent stimulation testing.

In a preferred embodiment, the system, based on an action label automatic classification function, is configured to perform standardized naming and identification for various actions executed by the user in daily activities, so as to avoid tremor determination confusion resulting from differences in user action naming, variations in posture execution pattern, or drift in identification algorithm. With this function, by integrating acceleration and angular velocity signals acquired by the IMU, key temporal features and spatial trajectory features of actions are extracted, the identified actions are automatically mapped to a standardized action library, and unified naming identifiers such as “pen grasping”, “drinking”, and “object holding” are assigned. Through this approach, the system may classify the action into an identical standardized action through feature clustering or feature similarity calculation, despite an identical action exhibits slight variations in diverse scenarios, so as to ensure consistency and comparability throughout the tremor analysis process. This strategy significantly reduces statistical biases arising from inconsistent action labels, ensures stable modeling of action-tremor relationships, and facilitates construction of generalizable stimulation parameter regulation strategies.

In a preferred embodiment, to mitigate impacts of sporadic errors, transient anomalies, or identification errors on action classification precision, the system may apply a sliding mean method or median filtering algorithm to smooth tremor amplitude data for each action category. Additionally, a multi-cycle consistency determination rule is set to ensure that only actions exhibiting a consistent tremor deviation trend in a plurality of independent acquisition cycles are incorporated into the candidate action set. During practical implementation, the system first filters action-tremor data sequences using a fixed window length to suppress interference from abrupt variations and transient noise on tremor level assessment. Subsequently, the system compares tremor data of the identical action in different time intervals. A tremor deviation is deemed statistically significant only when a stable trend of the action exhibiting a tremor level persistently higher or lower than the average tremor level is observed in a majority of the time intervals, and the action is identified as a “critical action” by the system. This strategy effectively enhances the robustness of the action screening mechanism, prevents sporadic errors from compromising action set construction, and improves the reliability of subsequent electrical stimulation parameter optimization.

In a preferred embodiment, the system incorporates an action frequency factor configured to measure an occurrence frequency of various actions in daily activities of the user, and assign different weights based on the occurrence frequency, such that screening of representative high-frequency actions exhibiting significant tremor variations is prioritized to constitute an action set. The core principle of this method lies in that higher-frequency actions are more likely to reflect primary scenarios of tremor interference and exhibit greater clinical intervention value. Consequently, such actions are prioritized during optimization of electrical stimulation strategies. During daily data acquisition phases, the system is configured to calculate occurrence frequency and duration for each action label, and construct an action frequency distribution model. In the process of action set screening, the system is configured to prioritize assessment of actions exhibiting both a high tremor fluctuation amplitude and a high occurrence frequency, and exclude those with low occurrence frequency, and particularly infrequent, non-typical actions. This strategy enhances the practicability and user experience of system intervention, enables the electrical stimulation strategy to better align with real-life scenarios, and reinforces the personalized features and long-term effects of the solution.

In a preferred embodiment, when the system detects actions exhibiting high instability in tremor manifestations in specific scenarios and characterized by significant magnitude fluctuations, frequent variations, or inconsistent responses, the system may temporarily exclude the actions corresponding to the scenarios based on stability assessment rules, so as to ensure the robustness and safety of subsequent stimulation parameter testing and selection processes. unstable tremor responses may originate from external interference, non-standardized action execution, or fluctuations in pathological conditions. Direct utilization of such data for parameter testing may lead to model determination errors, parameter mismatch, and even an elevated risk of adverse effects. The system is configured to automatically determine and temporarily exclude actions exhibiting high uncertainty by comprehensively determining historical stability assessment indices for action-tremor pairs, such as a tremor response variance, a response trend consistency, and threshold exceedance counts of standard deviation. Following subsequent data collection and trend improvement, the system may reassess whether the action is reincorporated into the action set. This mechanism effectively enhances controllability and result accuracy in stimulation testing processes, reduces interference from outliers on system determination, and improves the stability and safety of therapeutic strategies.

By constructing the action set based on tremor variations, the system may precisely assess tremor responses induced by actions under different stimulation conditions, and thus distinguishing sensitivity in the feedback regulation process is significantly enhanced. Compared with conventional methods that assess stimulation effects in a single static state, an action dimension is incorporated as an auxiliary variable for tremor manifestations, which not only improves the accuracy of parameter assessment but also enhances the adaptability of the system to a plurality of scenarios. Through a defined classification and action screening mechanism, the electrical stimulation system is capable of identifying stimulation parameter combinations with better therapeutic effects in challenging states, so as to achieve safe and effective individualized intervention.

Continuing with the above example of the patient with essential tremor, analysis of the system on daily data of the user reveals that the “horizontal arm raising” action exhibits an average tremor amplitude of 0.52 g, significantly higher than the average tremor level of 0.43 g, and is determined as a Category I action; the “fist clenching and releasing” action exhibits a tremor amplitude of 0.35 g, significantly lower than the average tremor level, and is determined as a Category II action; and the “wrist rotating” action exhibits a tremor amplitude of 0.44 g, showing no significant deviation from the average tremor level, and is not incorporated into the action set. Thus, an action set is constructed in the system, including two representative action categories, “horizontal arm raising” and “fist clenching and releasing”.

To more effectively assess the effects of electrical stimulation parameters in a plurality of motion states and ensure that selected parameters exhibit broad applicability and stability, it is required to design a continuous action sequence incorporating actions with different tremor-inducing features. Since the Category I actions readily induce or exacerbate tremor while the Category II actions exhibit slight or negligible tremor manifestations in natural states, alternating execution of these two action categories in a single testing procedure enables comprehensive observation of tremor response trends under unified stimulation parameter conditions, and thus screening of electrical stimulation parameters based on dynamic action states is achieved. This continuous action sequence serves as a standardized procedure for stimulation response assessment, which is helpful for unifying testing standards and enhancing accuracy in stimulation feedback regulation determination.

In the present disclosure, the continuous action sequence refers to an action sequence formed by combining a plurality of actions with different tremor response features in a predetermined order. The continuous action sequence may be either automatically generated by the system or progressively completed through user training.

After identifying the Category I actions and Category II actions incorporated in the action set, the system is configured to automatically select at least one representative action from each category and combine these actions sequentially according to a predetermined strategy to construct a complete continuous action sequence. The system may sequence these actions based on the inter-action coordination, execution difficulty, and physical load tolerance, such that the continuous action sequence is representative in an overall structure and easy to execute.

The constructed continuous action sequence includes at least one Category I action and at least one Category II action.

Preferably, the continuous action sequence includes alternately arranged Category I actions and Category II actions to ensure real-time performance and discernibility of stimulation feedback. In the testing procedure, the system is configured to guide the user to execute actions strictly according to the order of the continuous action sequence, and continuously monitor tremor manifestations throughout the entire action sequence to assess the suppression effects and adverse effect risks of the current electrical stimulation parameter set in different states.

In a preferred embodiment, the system, based on an action proficiency and a muscle strength distribution of the user, is configured to screen the Category I actions and the Category II actions being easier to execute and having a moderate action range to reduce execution barriers for the continuous action sequence.

In a preferred embodiment, the system incorporates an inter-action buffer phase, which allows the user to pause briefly during action transition, facilitates tremor detection and noise suppression, and enhances data stability.

In a preferred embodiment, the action order in the continuous action sequence may be dynamically optimized according to historical testing feedback. For example, Category I actions exhibiting the highest sensitivity to stimulation are prioritized at an initial position of the continuous action sequence to enable early-stage determination of whether the stimulation exceeds an acceptable range.

In a preferred embodiment, the system is configured to identify execution of the continuous action sequence in real time. Upon detection of action deviation or interruption, the system may automatically suspend stimulation output or prompt re-execution, so as to enhance interactive safety of the system.

By constructing the continuous action sequence including different action categories, the system is capable of dynamically assessing the stability and adaptability of stimulation effects under unified electrical stimulation parameter conditions. This method avoids the local optima problem caused by testing with static actions or in single scenarios, and enhances the generalization capability of the electrical stimulation solution for real-life scenarios. By utilizing the amplification effect of the Category I actions and the stable baseline of the Category II actions, comprehensive responses of stimulation to tremor fluctuations may be obtained within a short time to provide reliable support for subsequent parameter optimization and individual therapeutic effect tracking.

Continuing with the above example of the patient with essential tremor, the system identifies “horizontal arm raising” as a Category I action and “fist clenching and releasing” as a Category II action. The system constructs the following continuous action sequence based on action coordination and execution complexity: “horizontal arm raising”→“fist clenching and releasing”→“horizontal arm raising”→“fist clenching and releasing”, where each action lasts 10 seconds, and a transition interval between the consecutive actions is 2 seconds.

Because the intervention effect of electrical stimulation on tremor exhibits individual difference and action dependence, single parameters may prove effective for certain actions but incur adverse effects or become ineffective in other actions. Therefore, to enhance the universality and stability of stimulation parameters, it is necessary to test a plurality of candidate stimulation parameter sets in the continuous action sequence incorporating different tremor-inducing features. By guiding the user to sequentially execute the constructed continuous action sequence while applying the candidate stimulation parameter sets respectively, the system is configured to monitor tremor responses of each parameter set in different action states in real time, and screen optimal parameters that exhibit significant tremor suppression in the tremor-amplifying action and generate no adverse effect in the low-tremor action, and such optimal parameters are configured for subsequent electrical stimulation therapy and ensure the safety and efficacy of stimulation intervention in dynamic environments.

According to the constructed continuous action sequence, the system is configured to apply each candidate stimulation parameter set sequentially in each testing cycle, guide the user to execute the complete continuous action sequence, and meanwhile, acquire tremor amplitudes in each action phase via the IMU. Assessment standards are set in the system as follows: for Category I actions, the tremor level must be significantly lower than the historical average tremor level of the action under non-stimulation conditions; and for Category II actions, the tremor level cannot be significantly higher than the historical average tremor level of the action under non-stimulation conditions.

The system is configured to calculate and screen feedback effects of all candidate parameters in a plurality of testing cycles, and finally determine a first electrical stimulation parameter set that satisfies the above conditions. This parameter set is recorded in a parameter management module, and serves as default control parameters for subsequent electrical stimulation procedures.

To prevent transient fluctuations from affecting determination, the system preferably employs a sliding window or a median filtering approach for smoothing tremor data to enhance identification robustness.

In a preferred embodiment, based on an action proficiency and a muscle strength distribution of the user, the system is configured to screen the Category I actions and the Category II actions being easier to execute and having a moderate action range to construct a continuous action sequence. Consequently, physical and cognitive burdens of the user on execution of the continuous action sequence are reduced, and usability and adaptability of the system are enhanced. The action proficiency may be assessed by a completion duration, a postural stability, and an error occurrence frequency of each action in long-term IMU data, and the muscle strength distribution may combine an EMGs sensor to assess activation intensity of different muscle groups. During the action screening, the system may preferentially select actions frequently executed by the user in daily activities, exhibiting postural stability, and generating minimal execution errors, and exclude actions having high requirements for specific muscle groups or exhibiting difficulty in continuous completion. Through this approach, the constructed continuous action sequence may give consideration to tremor distinguishing capability and user executability; interruption, determination errors, or user discomfort caused by excessive action difficulty are avoided; and both integrity and efficacy of the electrical stimulation strategy in the assessment phase are enhanced.

In a preferred embodiment, the system incorporates an inter-action buffer phase in the continuous action sequence, which allows the user to pause briefly during action transition, such that the system may detect stability of tremor signals and suppress interference noise arising from action transition. This buffer phase is implemented as a preset short-term static window between consecutive action segments, typically lasting several seconds, and no active action is required for the user in the buffer phase. By utilizing low-amplitude motion data in the static window period, the system may fully acquire tremor responses triggered by the preceding action segment, and suppress high-frequency noise through a filtering algorithm to ensure that a clear and stabilized tremor amplitude profile is generated. Additionally, this buffer window is further configured to determine whether execution of the current action is completed and whether the next action is ready, and definite temporal boundaries are provided for subsequent effect attribution of stimulation parameters. This strategy significantly enhances the stability of data processing and the resolution of analysis, and prevents signal interference induced by action transition from compromising parameter determination results.

In a preferred embodiment, the system supports that the action order in the continuous action sequence is dynamically optimized according to historical testing feedback. For example, Category I actions exhibiting the highest sensitivity to stimulation are prioritized at an initial position of the sequence to enable rapid determination of whether the current stimulation parameters are appropriate. This optimization process is based on a stimulation-action response database acquired during prior testing. The system is configured to analyze the response amplitude, response delay, and adverse effect manifestations regarding tremor suppression effects of various action categories, and sequence the actions by sensitivity. During construction of the continuous action sequence, the system may select different sequencing strategies according to current testing objectives; for example, during testing of new parameter combinations, high-sensitivity actions are positioned at an initial position to enable rapid screening; during verification of safety, actions prone to inducing adverse effects are positioned at an end position to postpone the occurrence time of potential risks. This dynamic sequencing mechanism not only enhances the testing efficiency, but also improves the flexibility of the electrical stimulation feedback mechanism and the safety redundancy capability of the system.

In a preferred embodiment, the system is configured to identify execution of the continuous action sequence in real time. Upon detection of action deviation, interruption, or severe postural abnormality during execution, the system may automatically suspend the current stimulation output or send a prompt via an interface to guide the user to reexecute the corresponding action, so as to enhance interactive safety and control reliability of the system. During specific implementation, the system continuously monitors the execution state of the user through the action identification module, and matches the execution state with the preset standard action template. When action features in consecutive frames deviate from the standard template beyond a set threshold, or when IMU signals indicate abnormal states including action pause, directional reversal, or time exceedance, the system may immediately determine the occurrence of action distortion or interruption. To prevent inaccurate data from causing wrong stimulation determination, the system may automatically suspend subsequent stimulation output, which avoids adverse effects of ineffective or hazardous stimulation on the patient. This mechanism ensures the integrity of stimulation assessment and the availability of data, while enhancing the use confidence of the user and the safety of the system in clinical applications.

In this solution, action-inducing scenarios are incorporated as distinguishing conditions during selection of electrical stimulation parameters, and stimulation effects are assessed through cross-scenario tremor manifestations, such that the finally selected parameters have strong intervention capability and avoid new adverse effects arising from action transition, which significantly improves personalization, dynamic adaptability, and long-term stability of stimulation intervention. Compared with conventional static-feedback-based parameter selection approaches, this solution has higher safety, accuracy, and practicability, and is applicable to closed-loop therapeutic systems for action-associated tremor diseases including essential tremor and Parkinson's disease.

Continuing with the above example of the patient with essential tremor, totally five candidate stimulation parameter combinations are configured by varying stimulation frequencies and pulse widths in the system. When the user executes the continuous action sequence of “horizontal arm raising→first clenching and releasing→horizontal arm raising→clenching and releasing”, the system applies various parameter sets sequentially and records tremor responses. The results reveal that the third parameter set reduces the tremor amplitude from 0.52 g to 0.39 g during the “horizontal arm raising” phase, and maintains the tremor amplitude at 0.35 g without increase during the “fist clenching and releasing” phase, satisfies the set conditions, is determined by the system as the first electrical stimulation parameter set, and is recorded for subsequent electrical stimulation procedures. The remaining parameter sets fail to simultaneously satisfy the two conditions, and are consequently not adopted. Thereafter, the system prompts the user to initiate the standard electrical stimulation therapeutic procedure, and adopts this parameter set as a default output.

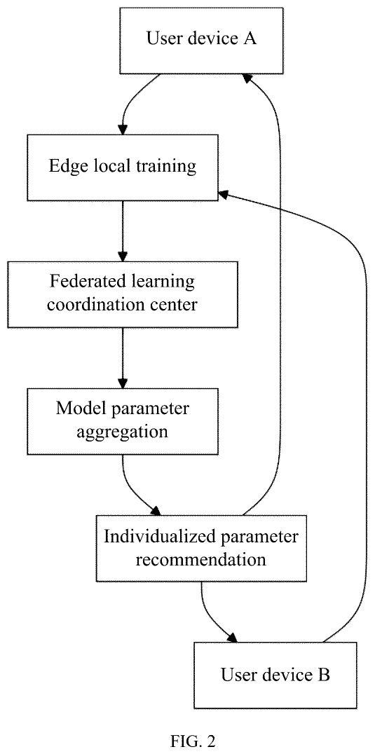

As shown in FIG. 2, in a preferred embodiment, the plurality of candidate stimulation parameter sets are determined by a cloud-based AI model in combination with historical data from a plurality of users.

To enhance the accuracy and adaptability of stimulation parameter settings, incorporation of a cloud-based AI model trained on large-scale historical user data during generation of the candidate stimulation parameter sets enables effective utilization of cross-user behavioral patterns and stimulation response patterns. Therefore, a parameter set with enhanced universality, efficacy, and individual adaptability is constructed. By aggregating nonlinear correlation information among tremor amplitude, electrical stimulation response, and action states from historical samples via the cloud-based model, the system is capable of generating predictive candidate parameter sets, thereby avoiding inefficiency and uncertainty caused by single empirical methods or random settings.

The plurality of candidate stimulation parameter sets refer to a plurality of electrical stimulation parameter combinations applied to a user during a testing procedure, typically including different configurations of stimulation frequency, pulse width, and voltage intensity. The cloud-based AI model refers to an artificial intelligence module deployed on a remote server. By performing multi-dimensional modeling on action data, tremor response data, and historically applied stimulation parameter results from a plurality of users, the cloud-based AI model may learn individualized response features between different individuals and stimulation.

In the specific implementation process, the system first acquires historical data samples from a plurality of users. These data samples include combined information of user action sequences, tremor variation trends, and corresponding electrical stimulation parameters. Based on the above data, the system constructs a prediction model using neural convolutional neural networks (CNN), recurrent neural networks (RNN), or graph neural networks (GCN); the input of the model is feature encoding and current action states of the user, and the output is the recommended stimulation parameter combination. The model is deployed to a cloud platform after being trained, and wearable devices request the model service via network to obtain a plurality of candidate stimulation parameter sets suitable for the current user.

In a preferred embodiment, the cloud-based AI model incorporates an individual adaptive weighting mechanism during the training phase to enhance recommendation precision of stimulation parameters for the current user. This mechanism, based on the individual difference modeling principle, is configured to identify historical data samples exhibiting high similarity to the target user in physiological parameters, action patterns, or tremor features, and assign higher learning weight to these samples during model training. For example, the system may screen a sample set exhibiting a similarity exceeding a preset threshold by calculating the similarity of EMGs mean values, IMU feature distribution and tremor frequency responses among users, and increase a weighting coefficient to at least twice that of normal samples. Through this approach, the model may be guided during the training process to focus on learning data features more representative of the current user state, such that the stimulation response trends and adaptation parameter ranges of the current user are predicted more accurately during the model inference phase. This mechanism elevates the individualization level of the model, reduces generalization error, and significantly enhances the personalized treatment capability of the electrical stimulation system.

In a preferred embodiment, to ensure the security of user privacy data and maintain the update capability of the AI model, the system is configured to construct a distributed collaborative training architecture by integrating a federated learning technology. In this architecture, each user terminal device is configured to perform local model training on personal physiological signal data, and updated model parameters (such as a weight matrix and a bias term) are encrypted by a local device and uploaded to the cloud platform, whereas raw physiological data are consistently retained on the local device of the user. Upon receiving local model parameters from a plurality of user terminals, the cloud platform integrates update information from different users through weighted averaging or dynamic aggregation algorithms, such that overall iteration and optimization of the AI model are completed. This mechanism achieves an effective equilibrium between global model performance and individual privacy protection, which not only reduces data leakage risks but also enhances model generalization performance and diversity adaptability. Additionally, this solution exhibits excellent scalability, and is applicable to continuous updating of personalized stimulation models in multi-terminal and multi-pathology scenarios.

In a preferred embodiment, the system is configured to sequence candidate stimulation parameter combinations outputted by the cloud-based AI model according to a prediction priority, and preferentially select high-confidence parameter sets for actual testing to reduce testing iterations and shorten parameter regulation time costs. This sequencing mechanism is based on confidence levels in effect prediction of the model for each parameter set in a given user state, typically manifested as probability scores of the output layer, or inverse indices of prediction errors. The system is configured to set a confidence threshold, define parameter sets exceeding the confidence threshold as a “high-priority candidate set”, and sequentially transmit such parameter sets to the stimulation module for verification testing. During testing, the system may terminate subsequent tests if ideal tremor suppression effects are obtained by using the first high-confidence parameter set, such that rapid parameter determination is achieved. This strategy ensures therapeutic efficacy, reduces user waiting time and testing redundancy, and effectively enhances response efficiency and user experience of the system, with particular applicability to scenarios requiring rapid response in clinical applications.

This strategy significantly enhances the efficiency and scientificity of parameter generation, provides more appropriate stimulation initiation parameter sets for individual users by fully utilizing tremor response commonality among different users, and improves the test success rate and therapeutic response speed. Additionally, the method exhibits dynamic updating and continuous optimization capabilities, and the recommendation capability is enhanced progressively through data accumulated as the system is utilized.

Exemplarily, the system may acquire, from the cloud-based AI model, five candidate stimulation parameter combinations trained on the basis of patients with analogous tremor patterns, and the candidate stimulation parameter combinations respectively range from (a frequency of 120 Hz, a pulse width of 90 μs, and a current of 1.5 mA) to (a frequency of 160 Hz, a pulse width of 120 μs, and a current of 2.0 mA). The model predicts that the first three parameter sets may be more effective in the tremor responses of the user, and consequently, testing priority is given to the first three parameter sets sequentially. In the testing procedure, the system finally selects the second parameter set as the first electrical stimulation parameter set based on action feedback, and the personalized closed-loop regulation is completed.

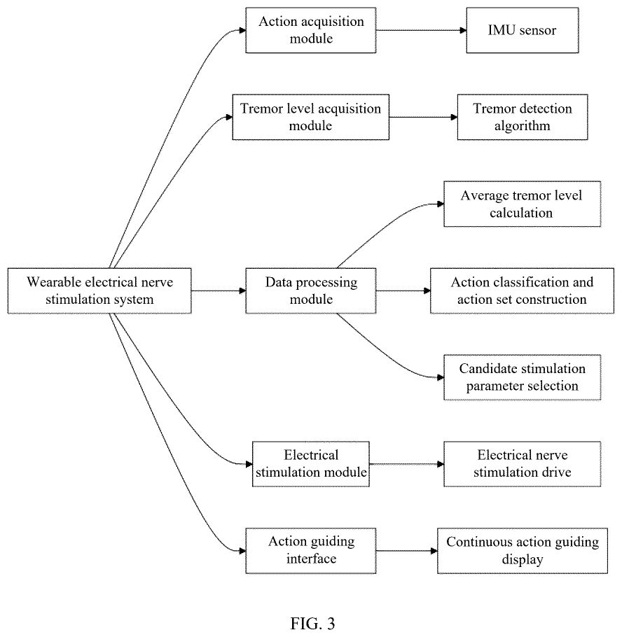

As shown in FIG. 3, in another embodiment, the present disclosure further provides a system for electrical stimulation based on physiological parameter feedback regulation, and the system includes:
a wearable electrical nerve stimulation system, where the system includes an electrical stimulation module, an action acquisition module, a tremor level acquisition module, and a processing module;
the action acquisition module is configured to continuously acquire action data in daily activities of a user;
the tremor level acquisition module is configured to synchronously acquire tremor manifestation data corresponding to the action data;
the processing module is configured to calculate an average tremor level of the user based on the tremor manifestations, identify a plurality of actions exhibiting tremor manifestations deviating from the average tremor level, and construct an action set that includes Category I actions having a tremor level significantly higher than the average tremor level, and Category II actions having a tremor level significantly lower than the average tremor level;
the processing module is further configured to construct, based on the action set, a continuous action sequence including at least one Category I action and at least one Category II action;
the electrical stimulation module is configured to respectively apply electrical nerve stimulation with a plurality of candidate electrical stimulation parameter sets when the user executes the continuous action sequence, and determine a first electrical stimulation parameter set; the first electrical stimulation parameter set satisfies the conditions that the tremor level of the Category I actions decreases, and the tremor level of the Category II actions does not rise; and
the electrical stimulation module is further configured to perform electrical nerve stimulation according to the first electrical stimulation parameter set during subsequent treatment.

In a further embodiment, user action signals are acquired via an IMU, temporal features of the IMU action signals are classified, and actions are classified based on the temporal features.

In a further embodiment, tremor level data are determined via a sliding window; a width of a statistical window is dynamically regulated based on a tremor signal fluctuation; the width of the statistical window is reduced when it is detected that the tremor signal fluctuation exceeds a first preset value; and the width of the statistical window is increased when the tremor signal fluctuation is below a second preset value.

In a further embodiment, the tremor level of each action category is smoothed by applying a moving average method or median filtering, and an action is incorporated into the action set only when the action exhibits a consistent deviating trend in a plurality of independent cycles.

In a further embodiment, the continuous action sequence includes alternately arranged Category I actions and Category II actions.

In a further embodiment, the Category I actions and the Category II actions are screened based on an action proficiency and a muscle strength distribution of the user.

In a further embodiment, an application order of candidate parameters is dynamically regulated based on a Bayesian optimization strategy.

In a further embodiment, the plurality of candidate stimulation parameter sets are determined by a cloud-based AI model in combination with historical data from a plurality of users.

It should be noted that the above explanation of the embodiments of the method for electrical stimulation based on physiological parameter feedback regulation is further applicable to devices in the embodiments of the present disclosure, which will not be repeated herein.

Those of ordinary skill in the art may recognize that units and algorithmic steps described in the embodiments disclosed herein may be implemented through electronic hardware, computer software, or a combination thereof. Whether these functions are executed in hardware or software depends on the particular applications and design constraints of the technical solution. Those skilled in the art may employ different methods to achieve the described functions for each specific application, but such implementation should not be construed as exceeding the scope of the present disclosure.

Those skilled in the art may clearly understand that for conciseness and clarity, the specific working processes of the described systems, devices, and units may be referenced to the corresponding processes in the embodiments of the above method, which will not be repeated herein.

In certain embodiments provided by the present disclosure, if any function is implemented in the form of software functional units and sold or used as an independent product, the function may be stored in one computer readable storage medium. Based on such understanding, the technical solution of the present disclosure may be embodied, either in essence or in the portion contributing to the prior art or in part of the technical solution, as a software product. This computer software product is stored in a storage medium, including some instructions for enabling one computer device (which may be a personal computer, server, network device and the like) to execute all or part of the steps of the method described in various embodiments of the present disclosure. The above storage medium includes various media that can store program codes, such as a USB flash disk, a mobile hard disk drive, a Read-Only Memory (ROM), a Random Access Memory (RAM), a magnetic disk or an optical disk.

The foregoing descriptions only represent specific embodiments of the present disclosure. Any alterations or substitutions readily conceivable by those skilled in the art within the technical scope disclosed herein should be construed as falling within the protection scope of the present disclosure. The module structures not explicitly defined in the present disclosure are subject to the contents recorded in the prior art. The prior art mentioned in the Background and Detailed Description sections of the present disclosure may be incorporated herein to understand the meanings of certain technical features or parameters.

What is claimed is:

1. A method for electrical stimulation based on physiological parameter feedback regulation, comprising:
providing a wearable electrical nerve stimulation system, wherein the system is configured to deliver electrical stimulation, and acquire action parameters and tremor level parameters;
continuously acquiring action data and corresponding tremor manifestation data in daily activities of a user;
calculating an average tremor level of the user based on the tremor manifestations;
identifying a plurality of actions exhibiting tremor manifestations deviating from the average tremor level, and constructing an action set, wherein the action set comprises Category I actions having a tremor level significantly higher than the average tremor level, and Category II actions having a tremor level significantly lower than the average tremor level;
constructing a continuous action sequence based on the action set, wherein the continuous action sequence comprises at least one Category I action and at least one Category II action; and
guiding the user to execute the continuous action sequence while respectively applying electrical nerve stimulation with a plurality of candidate stimulation parameter sets, determining a first electrical stimulation parameter set that satisfies the conditions that the tremor level of the Category I actions decreases relative to the daily level, and the tremor level of the Category II actions does not rise relative to the daily level, and performing subsequent electrical stimulation according to the first electrical stimulation parameter set.

2. The method for electrical stimulation based on physiological parameter feedback regulation according to claim 1, wherein user action signals are acquired via an inertial measurement unit (IMU), temporal features of the IMU action signals are classified, and actions are classified based on the temporal features.

3. The method for electrical stimulation based on physiological parameter feedback regulation according to claim 1, wherein tremor level data are determined via a sliding window; a width of a statistical window is dynamically regulated based on a tremor signal fluctuation; the width of the statistical window is reduced when it is detected that the tremor signal fluctuation exceeds a first preset value; and the width of the statistical window is increased when the tremor signal fluctuation is below a second preset value.

4. The method for electrical stimulation based on physiological parameter feedback regulation according to claim 1, wherein the tremor level of each action category is smoothed by applying a moving average method or median filtering, and an action is incorporated into the action set only when the action exhibits a consistent deviating trend in a plurality of independent cycles.

5. The method for electrical stimulation based on physiological parameter feedback regulation according to claim 1, wherein the continuous action sequence comprises alternately arranged Category I actions and Category II actions.

6. The method for electrical stimulation based on physiological parameter feedback regulation according to claim 1, wherein the Category I actions and the Category II actions are screened based on an action proficiency and a muscle strength distribution of the user.

7. The method for electrical stimulation based on physiological parameter feedback regulation according to claim 1, wherein an application order of candidate parameters is dynamically regulated based on a Bayesian optimization strategy.

8. The method for electrical stimulation based on physiological parameter feedback regulation according to claim 1, wherein the plurality of candidate stimulation parameter sets are determined by a cloud-based AI model in combination with historical data from a plurality of users.

9. A system for electrical stimulation based on physiological parameter feedback regulation, comprising:
a wearable electrical nerve stimulation system, wherein the system comprises an electrical stimulation module, an action acquisition module, a tremor level acquisition module, and a processing module;
the action acquisition module is configured to continuously acquire action data in daily activities of a user;
the tremor level acquisition module is configured to synchronously acquire tremor manifestation data corresponding to the action data;
the processing module is configured to calculate an average tremor level of the user based on the tremor manifestations, identify a plurality of actions exhibiting tremor manifestations deviating from the average tremor level, and construct an action set that comprises Category I actions having a tremor level significantly higher than the average tremor level, and Category II actions having a tremor level significantly lower than the average tremor level;
the processing module is further configured to construct, based on the action set, a continuous action sequence comprising at least one Category I action and at least one Category II action;
the electrical stimulation module is configured to respectively apply electrical nerve stimulation with a plurality of candidate electrical stimulation parameter sets when the user executes the continuous action sequence, and determine a first electrical stimulation parameter set; the first electrical stimulation parameter set satisfies the conditions that the tremor level of the Category I actions decreases, and the tremor level of the Category II actions does not rise; and
the electrical stimulation module is further configured to perform electrical nerve stimulation according to the first electrical stimulation parameter set during subsequent treatment.

10. The system for electrical stimulation based on physiological parameter feedback regulation according to claim 9, wherein user action signals are acquired via an inertial measurement unit (IMU), temporal features of the IMU action signals are classified, and actions are classified based on the temporal features.
